(19) United States
(12) Patent Application Publication
(10) Pub. No.: US 2026/0091142 A1
(43) Pub. Date: Apr. 2, 2026

(54) Glucose Responsive Color Changing Tattoo Ink

(71) Applicant: Austin Michael Clark, Folsom, CA (US)

(72) Inventor: Austin Michael Clark, Folsom, CA (US)

(73) Assignee: Amc.Tattoos LLC, Folsom, CA (US)

(21) Appl. No.: 18/904,094

(22) Filed: Oct. 2, 2024

(51) Int. Cl. A61K 49/00

(52) U.S. Cl. CPC A61K 49/0006; A61K 49/0017

(57) ABSTRACT

The present invention relates to a biocompatible tattoo ink that changes color in response to varying glucose levels in the bloodstream, providing a continuous monitoring method for individuals with diabetes. The ink contains glucose-sensitive agents that cause the tattoo to change from green (normal glucose levels) to red (high glucose levels) or blue (low glucose levels). Additionally, the ink shifts back to green when blood glucose returns to normal from either hyperglycemia or hypoglycemia. This provides a real-time visual indicator of glucose levels and enables users to monitor their health without additional equipment.

### BACKGROUND OF THE INVENTION

Diabetes management requires continuous monitoring of blood glucose levels to avoid dangerous complications associated with hyperglycemia (high blood sugar) and hypoglycemia (low blood sugar). Current glucose monitoring methods rely on devices that can be invasive, costly, or require frequent calibration. There is a growing need for a simple, intuitive system that can provide continuous monitoring of blood glucose levels.

Prior research has explored biosensors and glucose-responsive materials, but these technologies often suffer from limitations, including insufficient durability, slow response times, or difficulty in practical application. The present invention addresses these challenges by providing a biocompatible tattoo ink that changes color based on glucose levels, allowing for continuous monitoring and clear visual feedback.

### SUMMARY OF THE INVENTION

The invention relates to a glucose-responsive tattoo ink designed to provide continuous blood glucose monitoring. The ink is composed of a biocompatible carrier, glucose-sensitive agents, and stabilizing compounds. When blood glucose levels fluctuate, the ink changes color in real time:
At normal glucose levels, the tattoo remains green.
In the case of hyperglycemia (high glucose levels), the tattoo shifts to red.
During hypoglycemia (low glucose levels), the tattoo shifts to blue.
When glucose levels return to normal, the tattoo transitions back to green from either red (hyperglycemia) or blue (hypoglycemia).

This color-shifting mechanism offers a simple, clear, and real-time visual indicator of an individual's glucose status, allowing for improved glucose management without the need for invasive or cumbersome devices. The ink's formulation ensures long-term stability, biocompatibility, and reliable color responses to glucose concentration changes.

### DETAILED DESCRIPTION OF THE INVENTION

### Components of the Tattoo Ink

1. Biocompatible Carrier: The ink is formulated with a biocompatible carrier that is safe for use in the human body. The carrier allows for the smooth application of the tattoo and maintains the suspension of the glucose-sensitive agents. Suitable carriers include glycerin, distilled water, ethanol, or propylene glycol.

2. Glucose-Sensitive Agent: The key component of the ink is a glucose-sensitive agent that reacts with glucose levels in the interstitial fluid of the skin. When glucose levels rise or fall, the agent triggers a color change in the tattoo:
At normal glucose levels (normoglycemia), the agent causes the tattoo to appear green.
During hyperglycemia (high glucose), the agent induces a shift from green to red.
During hypoglycemia (low glucose), the agent induces a shift from green to blue.
When glucose levels return to normal, the agent reverses these changes, causing a transition from red or blue back to green.

3. Examples of glucose-sensitive agents include:
pH-sensitive dyes that change color due to local pH shifts caused by glucose metabolism.
Glucose oxidase enzyme, which reacts with glucose to generate gluconic acid and alter local pH.
Nanoparticles functionalized with glucose-binding molecules that cause reversible color changes based on glucose concentrations.

4. Stabilizing Agents: Stabilizing agents are added to the ink to prevent degradation of the glucose-sensitive agents and ensure long-term functionality. These stabilizers help maintain the ink's color responsiveness over time, ensuring accurate and reliable glucose monitoring.

### Color Change Mechanism

When the glucose-responsive tattoo ink is applied to the skin, it interacts with interstitial glucose, which reflects blood glucose levels. The ink's glucose-sensitive components react to varying glucose concentrations and trigger the following visible changes:

1. Normal Glucose Levels (Normoglycemia):
When glucose levels are within the normal range, the tattoo remains green.

2. Hyperglycemia (High Glucose Levels):
As blood glucose levels increase beyond the normal range, the glucose-sensitive agent reacts and induces a color shift from green to red.

3. Hypoglycemia (Low Glucose Levels):
When blood glucose levels drop below the normal range, the ink shifts from green to blue, indicating low glucose levels.

4. Return to Normoglycemia from Hyperglycemia:
When glucose levels drop from high to normal, the tattoo transitions back from red to green. This dynamic response helps users identify when their glucose levels have stabilized.

5. Return to Normoglycemia from Hypoglycemia:
Similarly, as glucose levels rise from low to normal, the ink shifts from blue back to green.

### Application Process

The glucose-responsive tattoo ink is applied using standard tattoo techniques, whereby the ink is injected into the dermal layer of the skin. The tattoo can be designed in any pattern and serves both a decorative and functional purpose. Once applied, the ink provides continuous glucose monitoring without the need for additional external devices.

### Manufacturing Process

1. Preparation of the Biocompatible Carrier:
The carrier is synthesized or prepared in a sterile environment, ensuring it meets medical-grade safety standards.

2. Incorporation of the Glucose-Sensitive Agent:
The glucose-sensitive agent (such as a pH-sensitive dye or glucose oxidase enzyme) is mixed into the biocompatible carrier to form a stable suspension.

3. Addition of Stabilizing Agents:
Stabilizing agents are incorporated into the formulation to preserve the reactivity and longevity of the glucose-sensitive components.

4. Sterilization:
The ink is sterilized to ensure it is free from contaminants before application to human skin.

1. A biocompatible tattoo ink composition comprising:
a biocompatible carrier medium;
a glucose-sensitive agent that changes color in response to glucose concentration levels in interstitial fluid, wherein the ink appears green at normal glucose levels, shifts to red at high glucose levels, and shifts to blue at low glucose levels;
stabilizing agents configured to maintain the stability and responsiveness of the glucose-sensitive agent over time.

2. The composition of claim 1, wherein the glucose-sensitive agent comprises a pH-sensitive dye that changes color based on local pH shifts caused by glucose oxidation.

3. The composition of claim 1, wherein the glucose-sensitive agent comprises glucose oxidase, which catalyzes a chemical reaction that induces a color change based on glucose levels.

4. The composition of claim 1, wherein the glucose-sensitive agent comprises a fluorescent biosensor that changes color based on glucose levels.

5. The composition of claim 1, wherein the biocompatible carrier is selected from the group consisting of glycerin, distilled water, ethanol, and propylene glycol.

6. A method for monitoring blood glucose levels in an individual, the method comprising:
applying the tattoo ink composition of claim 1 to the dermal layer of the skin;
observing the visible color change of the tattoo in response to blood glucose concentration changes;
interpreting the color change as an indication of blood glucose levels, wherein:
the color shifts from green to red in response to high glucose levels;
the color shifts from green to blue in response to low glucose levels;
and the color transitions back to green when blood glucose levels return to normal from either high or low glucose conditions.

7. The method of claim 6, wherein the color shifts from red to green when glucose levels return to normal from a hyperglycemic state.

8. The method of claim 6, wherein the color shifts from blue to green when glucose levels return to normal from a hypoglycemic state.
